(19) United States
(12) Patent Application Publication
(10) Pub. No.: US 2024/0281470 A1
(43) Pub. Date: Aug. 22, 2024

(54) N-HAMMING DISTANCE SEARCH AND N-HAMMING DISTANCE SEARCH INDEX

(71) Applicant: Voredos, Ramillies (BE)

(72) Inventor: Christian Felix Burckert, Saarbrucken (DE)

(73) Assignee: Voredos, Ramillies (BE)

(21) Appl. No.: 18/568,355

(22) PCT Filed: Jun. 28, 2021
(86) PCT No.: PCT/EP2021/067725

§ 371 Date: Dec. 8, 2023

(51) Int. Cl. G06F 16/901; G06F 16/903; G16B 30/10

(52) U.S. Cl. CPC G06F 16/9014; G06F 16/90344; G16B 30/10

(57) ABSTRACT

A method for performing an n-Hamming distance search is provided. The database comprises a plurality of records and an indexed storage. Each record comprises a record string associated with n+1 record hash values stored in an index. The method comprises partitioning a query string into n+1 query partitions. The n+1 query partitions are pairwise disjoint. A hash value is created for each query partition resulting in n+1 query hash values. Records having at least one record hash value equal to one of the n+1 query hash values are identified. The identified records are searched for records fulfilling a search condition. The search condition is that the record strings have a Hamming distance smaller than or equal to n with respect to the query string or a more limited search condition.

### BACKGROUND OF THE INVENTION

### A. Field of Invention

The present invention relates to a computer program, method, database and system for generating an n-Hamming search index and for performing an n-Hamming distant search.

### B. Description of the Related Art

Searching for strings in large datasets is a common problem. With the rise of Internet search engines like Google one may think the problem is generally solved. However, there is a big difference in strings that are built from words or texts, and arbitrary strings. The big difference is that words do not nearly cover the full space of possibilities a string generally provides. Therefore search-engines use e.g. word-vectors to efficiently search trillions of texts or words. It's clear, that word-vectors won't work with arbitrary strings (also called random strings), since they are not compounded of words. And not all strings do have equivalents for words. Sometimes the equivalents might exist but are hard to define. Generally, the structures in strings might be unknown, complicated, twisted or problematic in various reasons. Therefore, we use the notation of quasi-random strings. The idea is that strings are treated like fully random strings when designing the algorithm, but in its applications, they are not necessarily fully random.

Of course, picking quasi-random strings from a database is not a problem and can be easily solved using a simple hash index. However, the addressed problem arises when there are errors in the string. A simple hash cannot be used anymore to identify the quasi-random string because normal hash-indices are not error-stable. There are so called perceptual hashes but they address a different problem. Therefore, the search for quasi-random strings allowing a certain error is still mostly solved by a sequential comparison of each character of the string. Mostly the Levenshtein distance is used to define, when two strings are still similar. The Levenshtein distance measures the minimum number of single-character edits (insertions, deletions or substitutions) required to change one string sequence into the other. All search strings having a Levenshtein distance smaller than a certain threshold are output as result of the error allowing search. The computation time of a calculation of a Levenshtein distance scales at least with the length l of the longer string to be compared. So, the search in a database with m entries would scale with O(m*l).

One application in which this problem becomes very important is the search for biological sequences, because the sequences are very long and/or the databases comprises often billions of records to be compared. Biological sequences like nucleotide sequences or amino acid sequences can be treated as quasi-random strings. Nucleotide sequences are normally very long and have an alphabet of at least 4 nucleotide symbols. Amino acid sequences or proteins are normally shorter and have an alphabet of at least 20 amino acid symbols.

One biological example application is the search for proteins based on their complementary-determining region (CDR). Immunoglobulins and T-cell receptors have CDRs. There are six CDR for each antigen receptor that can collectively come into contact with the antigen. The six CDRs contain normally three heavy chain CDR (CDR-H1, CDR-H2, CDR-H3) and three light chain CDR (CDR-L1, CDR-L2, CDR-L3). A very important research tool is to search for protein peptides with identical or similar CDRs. Very often it is looked for similar CDR-H3, but it could also be important to look for a similar combination of different CDRs like a similar CDR-H3 and CDR-H1 or CDR-H2. The peptide strings of the CDRs are normally only between 10 and 30 characters long, longer and shorter variants exist. However, since the databases contain often several billions of records, a similarity search for a protein peptide string in a CDR-H3 database requires often several hours even at state-of-the-art high processing machines.

The DNA sequences provide many different search problems which are quite different from the search problems of amino acid sequences. One solution often used for comparing DNA sequences are hash values. For example, in US20200135298, read collapsing is performed by identifying similar DNA reads with locality sensitive hashing. Since the hash values are compared instead of their individual characters, similar sequence reads can be identified much quicker than with a character comparison allowing a fast classification of the reads. In US20150112884, genetic relatives are identified without compromising its privacy by sub-grouping the compared DNA sequences and comparing the hash values of the sub-groups. The number of identical hashes is a sign for the similarity. In CN111370064, a blast algorithm is proposed which calculates the hash values of all k-mers of the sequences to compare and counts the number of identical hash values. U.S. Pat. No. 8,943,091 describes a database structure optimized for searching strings storing sub-portions It is proposed to use a hash index storing the hashes of all k-mers of the record strings or to store disjoint sub-portions in an FM index. However, these solutions do not allow a fast search with a defined string distance In addition, these solutions are not advantageous when it comes to shorter strings as the hashes for the strings and their sub-strings become longer than the hashes themselves. So, these technologies are not able to speed up the search of CDR peptide strings with a length between 10 and 30 characters in a very large database.

Some embodiments of the present invention may provide one or more benefits or advantages over the prior art.

### SUMMARY OF THE INVENTION

It is the object of the invention to provide a universal search technology which allows a string search with a defined allowable maximum string distance at high speed.

The object is solved by a method for performing an n-Hamming distant search in a database, wherein the database comprises a plurality of records and an indexed storage, wherein each record comprises a record string, wherein each record of the database is associated to n+1 record hash values stored in an index of the indexed storage, wherein the method comprises the following steps: partitioning a query string into n+1 query partitions, wherein the n+1 query partitions are pairwise disjoint; creating a hash value for each query partition resulting in n+1 query hash values; identifying records having at least one record hash value equal to one of the n+1 query hash values resulting in identified records; and searching within the identified records for resulting records fulfilling a search condition, wherein the search condition is that the record strings of the resulting records have a Hamming distance smaller than or equal to n with respect to the query string or a more limited search condition.

The object is solved by a method for generating an n-Hamming search index for a database allowing an n-Hamming distant search in the database, the method comprises for each record of the database the following steps: partitioning the record string into n+1 record partitions, wherein the n+1 record partitions are pairwise disjoint; creating a hash value for each record partition resulting in n+1 record hash values; and storing the n+1 record hash values as n-Hamming search index value for the record in the n-Hamming search index.

The object is solved by a computer program (transitory or non-transitory) comprising instructions, when executed on a processor, configured to perform in the processor the steps of one of the previously described methods.

The object is solved by a database with an n-Hamming search index allowing an n-Hamming distant search, the database comprising a plurality of records, wherein each record comprises a record string and an n-Hamming search index value, wherein the n-Hamming search index value of the respective record comprises n+1 record hash values, wherein the n+1 record hash values correspond to the hash values of n+1 partitions of the record string of the respective record, wherein the n+1 partitions of the record string of the respective record are pairwise disjoint, wherein n-Hamming search index is constituted by the n-Hamming search index values of the records.

The object is solved by a system comprising a data storage for storing a database according to the previous claim and a processor configured to perform a search in the records of the database according to the method described above.

The fact that the partitions are disjoint and comprise (at least) one more partition than allowed errors in the n-Hamming distant search, the search space can be significantly reduced by an indexed search in the n-Hamming search index based on the n+1 hash values of the n+1 partitions of the record strings. If a record has no record partition equal to the n+1 query partitions of the query string (and thus no equal hash value), it is clear that the record string must have at least n+1 errors with respect to the query string and the record can be discarded. This search space reduction allows to perform similarity searches based on a Hamilton distance in very large databases in seconds instead of in hours as is the case when the record strings of each record are compared to the query string. A further advantage of the present search is that the result is not only fast, but also exact. The search will give out all records fulfilling the search condition and not just a rough estimate for similar strings.

The dependent claims refer to further advantageous embodiments of the invention.

In one embodiment, a user can select or program different search conditions as the more limited search condition. This allows to define within all the records of the database having a Hamming distance smaller than or equal to n more limited search conditions according to the needs of the user. All further conditions can be checked more or less at the same time as the main condition, as the same search space reduction applies.

In one embodiment, the searching within the identified records for resulting records is performed with an algorithm which works on the record strings of the identified records to verify the search condition. While the identification of the identified records works based on the n-Hamming search index, i.e. on the n+1 hash indices, (indexed search) and can thus be performed very rapidly notwithstanding the high number of records. The identified records are normally only a small percentage of the totality of records of the database, so that the search among the identified records for the resulting records fulfilling the search condition can be performed quickly as well, even if this search is based on the record string.

In one embodiment, the alphabet of the query string and the record strings comprises twenty or more different symbols.

In one embodiment, the length of the record strings is smaller than hundred characters.

The n-Hamming search according to the invention is particularly advantageous for short record/query strings and for alphabets with a high number of symbols.

In one embodiment, the query string is partitioned such that at least one of the n+1 partitions comprises a non-consecutive character sequence of the query string. This has the advantage that pseudo-random strings which tend to have similar sub-sequences lead still not to a high number of identical partitions as the partitions are shuffled. Due to the reduced number of collisions by equal partitions, the method can be performed even faster. But for fully random data, such a non-consecutive partition function is not necessary and any other partition function can be used. An example for such pseudo-random strings with a tendency to have similar start and end sequences are CDR-H3 sequences of proteins

In one embodiment, the hash value for each partition is created by applying a hash function on the combination of the partition and a salt information. An example for the salt information is one or more of the lengths of the query string, the lengths of the partitions of the query strings, an identifier of the content type of the query string and an identifier of the partition on which the hash is applied. The salt information allows to increase the length of the hashed data so that the risk of collision for short partitions is reduced and such the speed of the identification/search space reduction step is accelerated.

In one embodiment, the step of identifying records having at least one record hash value equal to one of the n+1 query hash values resulting in identified records corresponds to identifying records having for at least one i being a natural number between 1 and n+1 the i-th hash value of the record string equal to the i-th hash value of the query string as the identified records. By comparing always the respective partitions/hash values, the number of collisions is further reduced and the identification/search space reduction step is further accelerated.

In one embodiment, the identified records are identified as the records cumulatively having at least one record hash value equal to one of the n+1 query hash values resulting in identified records and having the same length as the query string. Since the Hamming distant search allows only searches of equal length, the search space reduction based on the length reduces further the search space and can thus accelerated the search.

In one embodiment, the records of the database are stored in an indexed storage with the n+1 hash values of the records working as indices for the indexed storage, wherein the identified records are identified by searching the n+1 query hash values in the respective n+1 hash value indices.

In one embodiment, the length of the record string is a further index of the indexed storage.

In one embodiment, each record comprises a Protein.

In one embodiment, the record string is an amino acid sequence.

In one embodiment, the record string is a complementarity-determining region of a protein.

In one embodiment, the system comprises an internal storage area configured, when the processor runs the search, to store the entire index of the database with the n+1 hash values of all records of the database.

Other benefits and advantages will become apparent to those skilled in the art to which it pertains upon reading and understanding of the following detailed specification.

### BRIEF DESCRIPTION OF THE DRAWINGS

FIG. 1 shows a query string.

FIG. 2 shows an embodiment of a database with a plurality of record strings.

FIG. 3 shows a first embodiment of a partition function.

FIG. 4 shows a second embodiment of a partition function.

FIG. 5 shows a first embodiment of an indexed storage of the database of FIG. 2.

FIG. 6 shows a second embodiment of an indexed storage of a database storing a plurality of record strings.

FIG. 7 shows the steps of computing k hash values of a string.

FIG. 8 shows the steps of generating an n-Hamming search index for a record in a database.

FIG. 9 shows the step of performing an n-Hamming distant search for a query string in a database with an n-Hamming search index.

FIG. 10 shows an exemplary system for performing an n-Hamming distant search.

FIG. 11 illustrates the step of identifying records based on the hashes with the example of query string of FIG. 1 and the database of FIG. 2.

FIG. 12 illustrates the step of identifying records based on the hashes and the length with the example of query string of FIG. 1 and the database of FIG. 2.

FIG. 13 illustrates the step of searching among the identified records for the records fulfilling an n-Hamming distance with respect to the query string with the example of query string of FIG. 1 and the database of FIG. 2.

In the drawings, the same reference numbers have been allocated to the same or analogue element.

### DETAILED DESCRIPTION OF THE INVENTION

Other characteristics and advantages of the present invention will be derived from the non-limitative following description, and by making reference to the drawings and the examples.

Before explaining the invention, we would like to define a number of terms used herein.

A string is a sequence of characters. The length of the string is defined by the number of characters contained in the sequence of characters. Each character comprises an element/symbol of an alphabet. The alphabet is a set of symbols. Each character of the string comprises one of the symbols of the alphabet. All the characters of the strings comprise symbols of the same alphabet. Strings related to the same alphabet are strings whose characters comprise (only) symbols of the same alphabet. The invention is applicable for any alphabet. The alphabet can comprise letters, digits, nucleotides, amino acids or any other symbols. The invention is particularly advantageous for alphabets with or more symbols, preferably with 10 or more symbols, preferably with 15 or more symbols, preferably with 16 or more symbols, preferably with 17 or more symbols, preferably with 18 or more symbols, preferably with 19 or more symbols, preferably with 20 or more symbols. Some alphabets comprise a combination of direct symbols and indirect symbols. Direct symbols can have only one meaning, while indirect symbols can have the meaning of at least two direct symbols. An indirect symbol could be a first direct symbol or a second direct symbol. Another indirect symbol could be not a first direct symbol. Another indirect symbol could be any of the indirect symbols. Preferably, the alphabet has more than 5, preferably more than 6, preferably more than 7, preferably more than 8 direct symbols. The invention is particularly advantageous for biological sequences like nucleotide sequences and amino acid sequences, in particular for the latter.

One possible symbol format for nucleotide sequences and amino acid sequences is the FASTA format. However, other formats are also possible. Strings of nucleotide sequences contain nucleotides as symbols of the alphabet. The alphabet for nucleotide sequences comprises at least four (direct) symbols, an Adenine (A), a Cytosine (C), Guanine (G) and Thymine (T) or Uracil (U) from which the characters of the string or nucleotide sequence can be chosen. For DNA sequences, the (direct) symbols of the alphabet would be ACGT and for RNA sequences, the (direct) symbols of the alphabet would be ACGU. The letters in parenthesis represent the nucleotide in the single letter annotation. Obviously, other representation/annotation for the nucleotide can be used. Strings of amino acid sequences, often also called protein peptides, contain amino acids as symbols of the alphabet. The alphabet for amino acid sequences comprises at least twenty (direct) symbols: Alanine (A), Cysteine (C), Aspartic acid (D), Glutamic acid (E), Phenylalanine (F), Glycine (G), Histidine (H), Isoleucine (I), Lysine (K), Leucine (L), Asparagine (M), Proline (P), Glutamine (Q), Arginine (R), Serine (S), Threonine (T), Valine (V), Tryptophan (W), Tyrosine (Y) from which the characters of the string or amino acid sequence can be chosen. The alphabet for amino acid sequences can optionally comprise further one or more of the following amino acids and/or (direct) symbols: Pyrrolysine (rare) (O), Methionine/Start codon (M), Selenocysteine (rare) (U) and stop codon (X). The letters in parenthesis represent the amino acids in the FASTA format. Obviously, the invention can also be applied for any other types of strings, characters and alphabets.

A character is defined by its position in the string and its symbol (of the alphabet). The position of a character in a string defines where the character is positioned in the sequence of the characters of the string. Thus, two characters of a string having the same symbol, but different positions are different as they distinguish in their positions. The character sequence of the string has preferably a first position defining the position of the first character of the (character sequence of the) string. The character sequence of the string bas preferably a last position defining the position of the last character of the (character sequence of the) string. Since the string has a well-defined character sequence, the order of the characters is important, and the positions of the characters follow a consecutive order. As will be explained in more detail below, it is also possible that the consecutive order is defined in a different way and/or is configurable. A consecutive character subset of a string is any subset of characters of the string having the same order as in the string. A non-consecutive character subset of a string is any subset of characters of the string having not the same order as in the string, i.e. having positions which are not consecutive. For example, a string ABCDEF has character A at position 1, character B at position 2 and so on. The exemplary character subsets ABC, BCDE, CD, DEF would be consecutive character subsets from the consecutive positions 1 to 3, 2 to 5, 3 to 4, 4 to 6, respectively. The exemplary character subsets ACE, BCE, CF would be non-consecutive character subsets from the non-consecutive positions (1,3,5), (2,3,5), (3,6), respectively.

A partition of a string is a character subset of the string. Preferably, the partition of a string is a proper subset of the string, i.e. the partition has a length smaller than the string. Two partitions of a (same) string are disjoint, if the character subsets of the two partitions do not overlap, i.e. if the intersection of the two partitions is empty. A plurality of partitions of a (same) string are disjoint, if all partitions are pairwise disjoint, i.e. if the intersection of the plurality of partitions is empty. In other words, each character of the string is element of only one partition. A plurality of partitions of a string constitutes the string, when the union of the plurality of partitions yield again the string.

We define here two metrics to measure a distance between two strings. A Hamming distance is defined by the number of positions at which the corresponding symbols of the two strings are different. The Hamming distance is defined only for strings of the same length. A distance allowing also inserts and deletes is the Levenshtein distance which thus works also for strings of different length. The Levenshtein distance measures the minimum number of single-character edits (being insertions, deletions or substitutions) required to change one string sequence into the other.

An n-distant search in the database is a search which gives out record strings of the plurality of record strings of the database having a distance smaller than or equal to n with respect to a query string.

An n-Levenshtein-distant search is an n-distant search wherein the distance n of the n-distant search is defined by the Levenshtein distance. In other words, the n-Levenshtein distant search is a search which gives out record strings of a plurality of record strings having a Levenshtein distance smaller than or equal to n with respect to a query string.

An n-Hamming-distant search is an n-distant search wherein the distance n of the n-distant search is defined by the Hamming distance. In other words, the n-Hamming distant search is a search which gives out record strings of a plurality of record strings having a Hamming distance equal to n with respect to a query string. Thus, an n-Hamming distant search gives out only record strings of the same length than the query string, because the Hamming distance is only defined for strings of equal length.

The n-Hamming distant search according to the invention allows to search for (records with) record strings having a Hamming distance smaller than or equal to n with respect to the query string. In one embodiment, the n-Hamming distant search is a search which gives out all record strings of the plurality of record strings of the database having a Hamming distance smaller than or equal to n with respect to a query string (not restricted search). This is the broadest possible search of a n-Hamming distant search using an n-Hamming search index. In another embodiment, the n-Hamming distant search in the database is a search which gives out all record strings of the plurality of record strings of the database having a Hamming distance smaller than or equal to n and fulfilling a further search condition. In other words, the n-distant search in the database of this embodiment is a search which gives out all record strings of the plurality of record strings of the database fulfilling cumulatively a first condition and a second condition, wherein the first condition is that the record string has a Hamming distance smaller than or equal to n, and the second condition corresponds to the further search condition. The further search condition is preferably a condition which truly restricts the search to less results (at least theoretically). The further search condition could be for example that the record strings given out by the search have only a Hamming distance smaller than or equal to a value I with respect to the query string, wherein I being smaller than n. Thus, all record strings having a Hamming distance between l+1 and n would not be given out by such a n-Hamming distant search with this further search condition. Like this, any 1-Hamming distant search with I being smaller than n can be realised by the further search condition. Preferably, the further search condition can be selected or configured by the user of the search. An example for a search condition which does not truly restricts the search would be for example the length of the query string since this is already intrinsic with a search for any Hamming distance. Thus, such a further search condition would never lead to any reduction of the records given out by the n-Hamming distant search.

A database comprises a plurality of records. Each record comprises at least one string which is subsequently called record string. So, a database comprises a plurality of record strings. A database might comprise two or more (different) record strings per record. Each record of the database might comprise a first record string and a second string. Each record of the database might comprise a first record string, a second record string and a third record string. In a preferred embodiment, the record string is an amino acid, preferably a CDR, preferably a CDR-H, preferably a CDR-H3. Preferably, the database comprises in the first, second and third record string three different CDRs of a protein. Preferably, the database comprises in the first record string a CDR-H1, in the second record string a CDR-H2 and in the third record string a CDR-H3. The database could be arranged as table with the records being rows and the record content might be written in one or more columns of the respective row. A first column (not limitative for the position of the column in the table) could contain the first record string of each record. A second column (not limitative for the position of the column in the table) could contain the second record string of each record. A third column (not limitative for the position of the column in the table) could contain the third record string of each record. Further columns could comprise an index to search through the records. The (first) record strings of all records are preferably strings related to the same alphabet. The second record strings of all records are preferably strings related to the same alphabet. The third record strings of all records are preferably strings related to the same alphabet. The first, second and third record strings of all records are preferably strings related to the same alphabet. When some record strings contain n or less non-alphabet characters, the method and/or system according to the invention would also still work, because they are simply considered as mismatch. The database comprises preferably more than 1 million records, preferably more than 10 million records, preferably more than 100 million records, preferably more then 500 million records, preferably more than 1 billion records. The number of records stored in the database is herein also abbreviated as M.

An indexed database comprises at least one indexed storage. The indexed storage comprises at least one index. Each record of the database has an index value. The index values of all records associated with their records are stored in the index of the indexed storage of the indexed database. The index of the indexed storage allows to search for an index value in less than O(M) The indexed storage can be a hash table, a tree or something else ordered based on the index values. The tree can be for example a btree. The index value of a record string of a record is retrievable/computable from the record string itself. E.g. a hash value or the length of the record string. When searching for a record with a certain index value, it is not necessary to check all records one after the other, but it is sufficient to find the index value in the indexed storage and check the associated record(s). The index can comprise a plurality of sub-indices. Preferably, the database allows to logically combine searches within at least two, preferably all of the plurality of sub-indices, e.g. by OR or AND or other logical operators. The database can store the complete records in the indexed storage However, preferably the indexed storage contains for each index value associated with one or more records, just a pointer or any other link to the storage of the one or more record(s) associated with the index value. The indexed database can for example be a relational database, a cluster-database or any other indexed database. The indexed of the indexed storage should have indices for range queries like hash-indexes, btrees, etc.

A hash value of a character sequence is a value obtained by applying a hash function on the character sequence. A hash function is any function that can be used to the character sequence of arbitrary size to a hash value. The hash value is preferably of fixed size independently of the size/length of the character sequence on which the hash function is applied. The hash value of a string is the hash value resulting from applying the hash function on the character sequence of the string. The hash value of a partition is the hash value resulting from applying the hash function on the character sequence of the partition. A salted hash value of a character sequence is the application of the hash function on a well-defined combination of the character sequence and a salt information. The combination can for example be a concatenation of the character sequence and a salt information or any other defined mixture of the character sequence and a salt information. A hash value of a character sequence can be a pure hash value of the character sequence or a salted hash value of the character sequence. Hash values are often used as index for an indexed storage, e.g. in a hash table.

A collision is when the same index value is used for several records. This can appear, because the records have the same data underlying the index value, e.g. the same character sequence of the partition underlying the hash value of the partition. This can further appear, because the function for calculating the index value, preferably a hash function, yields for two different character sequences (underlying the index value/hash value) of two records the same index/hash value.

To explain the basic concept of the invention, let's assume a simple example n-Hamming distant search with a fix Hamming-distance of n=2 and take a look at the following record string:
ABCDEFGHI

The 2 errors can be everywhere and it's hard to address the problem. But let's split the record string into 3 disjoint partitions:
ABC|DEF|GHI

If you have two errors, in the worst-case, they can only be found in 2 of the 3 partitions. So, one of the partitions does not contain an error. And this partition can be used to reduce the search-space. Each partition is hashed and these hash values h1=hash(ABC), h2=hash(DEF), h3=hash(GHI) are stored in an indexed storage for example with known indices like btrees or hash-indices. When you now want to search for a query string s with 2 errors you partition it into 3 partitions and query the three with a search query identifying all record strings having at least one of the three hashes equal to a corresponding at least one of the three hashes of the query string. Since the 2 errors can only appear in 2 partitions, at least one partition will be correct. By OR-ing the result the correct ones will be found.

Preferably, the length of the string is stored in an indexed storage like a btree as well. This allows to include the length of the query string as well in the search query. Since the n-Hamming distant search allows only to search for strings of identical length, all record strings with a length other than the length of the query string can thus be excluded from the search and the search space can be efficiently reduced. So, a 2-distant Hamming search for a query string of length l in a database would thus identify each record string having the length l and having at least one hash of the 3 partitions of the respective record string equal to at least one hash of the 3 partitions of the query string.

The indexed storage will use its indices on h1, h2, h3 and l to reduce the search-space. All indices might contain false positives, i.e. the wrong partitions and/or possible hash-collisions. Therefore, a further search needs to be performed after the search space reduction.

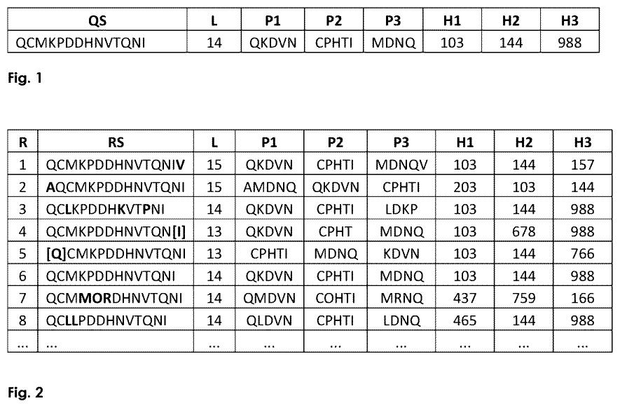

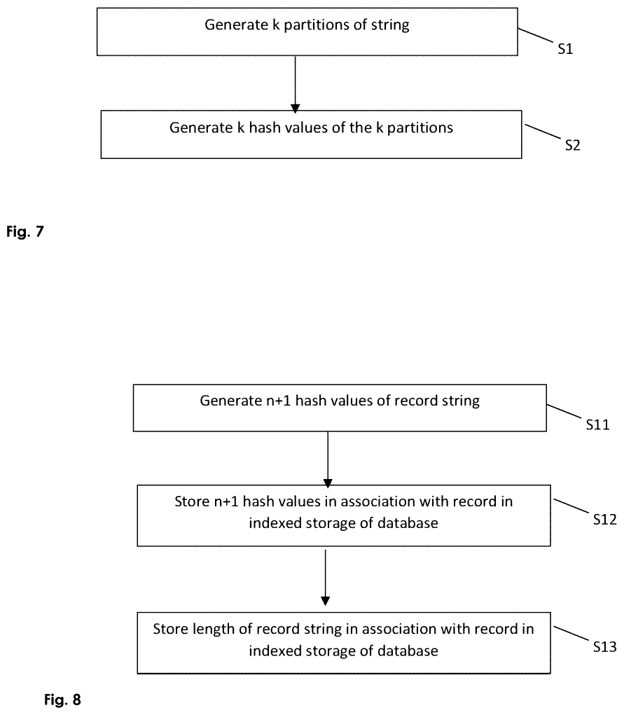

Before explaining the n-Hamming distant search, it will be explained with the help of FIG. 7 the steps to compute k hash values of a string. This will be explained at the example of the string QS of FIG. 1 with k=3. Obviously, the subsequent explanation applies equally for any other string (e.g. RS) or any other k.

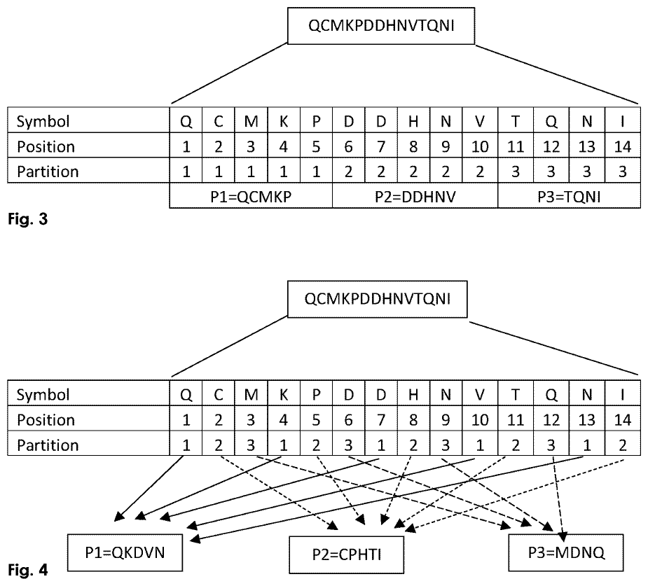

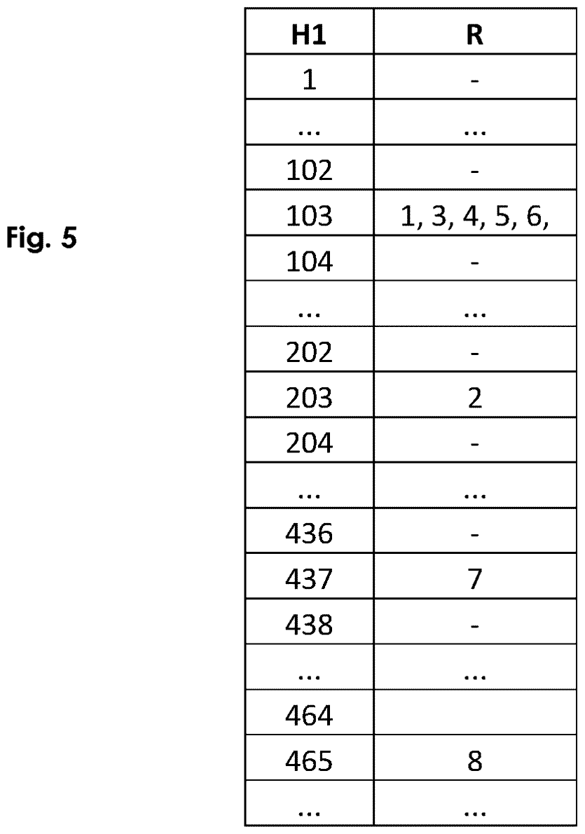

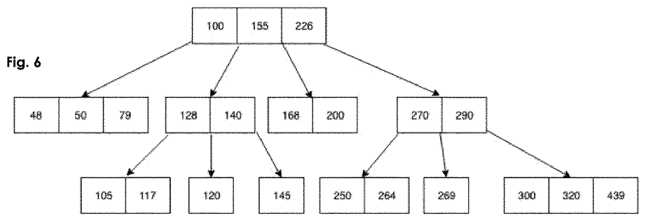

In a step S1, the string QS is partitioned into k partitions. The string QS is preferably partitioned into k partitions based on a partition function. The partition function and/or the k-partitions is/are preferably such that the k partitions of the string QS are disjoint. The partition function and/or the k-partitions is/are preferably such that the k partitions constitute the string QS. The partition function and/or the k-partitions is/are preferably such that the k partitions have a length difference of at most 1, i.e. the k partitions are substantially of equal length. If one partition is significantly shorter than the others, the number of collisions of the hash values of the shortest partition will increase with respect to the longer partitions. Therefore, it is best to balance the length of the k partitions. If the length of the string QS is not dividable by k, one or more of the partitions are longer by 1 character than the shortest partition. The most obvious partition function divides the string in k consecutive partitions. For (purely) random strings, such a partition function is perfectly fine. FIG. 3 shows an example for such a consecutive partition function for the string QS of FIG. 1 with k=3 into 3 partitions P1, P2 and P3. However, for pseudo-random strings which might have a tendency to have at the beginning and/or at the end often similar character sub-sequences, it would reduce the number of bash collisions, when the partitions are non-consecutive. Therefore, the partition function and/or the k-partitions is/are preferably such that at least one of the k partitions, preferably all k partitions is/are non-consecutive (non-consecutive partition function or non-consecutive partition(s)). This shuffles the characters of the string among different partitions and reduces the risk of collisions for certain pseudo-random strings like for amino acid sequences, in particular for CDR peptide strings. FIG. 4 shows such a non-consecutive partition function for the string QS of FIG. 1 with k=3 into 3 partitions P1, P2 and P3. Here the partition function is simply the modulo k function, i.e. all the characters at the position i+k*x (with x=0, 1, 2, . . . ) go into the partition i modulo k. However, many other non-consecutive partition functions would work equally well. The example string QS is partitioned with the partition function of FIG. 4 into k=3 partitions P1, P2 and P3. The string QS-QCMKPDDHNVTQNI has been partitioned in FIG. 1 using the partition function described in FIG. 4 into 3 partitions P1=QKDVN, P2=CPHTI, P3=MDNQ.

In a step S2, the hash values of the k partitions are calculated. K hash values are calculated with one hash value for each of the k partitions. Preferably, the i-th hash value Hi is calculated by applying the hash function on the i-th partition Pi, with i=1, 2, . . . , k. Preferably, the same hash function is used for each of the k partitions. However, it is theoretically also possible to use different hash functions for different partitions. It is only important to use always the same hash function for the same, e.g. the i-th, partition for each record string and each query string of the same database. The hash values resulting from the hash function have probably a (fixed) binary length, preferably a number being an n-th power of 2, i.e. 2, 4, 8, 16, 32, 64. The binary length of the hash value is one parameter for determining the probability of collisions between different character sequences resulting in the same hash value. Preferably, the hash value is 16 bits long or longer. Preferably, the hash value is 32 bits long or longer. In one embodiment, the hash value is 64 bits long or longer. A hash value of 64 bits results in approximately 1.8*10^19 different potential hash values which practically excludes the appearance of a collision. However, this increases the storage space for the indexed storage significantly. A hash value with 32 bits results in approximately 4.2 billion potential hash values which will probably provide multiple collisions in a database with a billion entries. This might reduce the speed of the search a bit but reduces also the storage space needed for the indexed storage. The size of the hash value must be chosen for the specific application to find the best trade-off between speed and storage space. In FIG. 1, the 3 hash values H1=hash(P1), H2=hash(P2), H3=hash(P3) are calculated for the 3 partitions P1, P2, P3, wherein hash() being the hash function. For exemplary reasons, an imaginary hash function has been chosen with a hash value of binary length 10 illustrated as a decimal number between 1 and 1024 for illustration purposes only.

In one embodiment, a salted hash function is used to calculate the hash functions. As a salt information, the length of the string QS, the length of the partition(s), an identifier of the record string (e.g. its column title), an identifier of the partition being hashed etc. can be used. This reduces the number of collisions for small strings (strings with a small or even zero length) with consequently small partitions. For an application with a first record string and a second record string in the same record and the identifier of the record string as salt information, the identifier of the first record string is added as salt information to a/any partition of the first record string so that the hash function is applied on a mixture on the combination of the partition of the first record string and the identification of the first record string, and the identifier of the second record string is added as salt information to a/any partition of the second record string so that the hash function is applied on a mixture on the combination of the partition of the second record string and the identifier of the second record string. Thus, partitions of the first and second record string having the same character sequence produce still different hash values so that they could be stored even in the same hash index. For an application using the identifier of the partition as salt information, partitions with different partition identifiers but equal character sequences will not have any more the same hash values. This might allow to store different partition hash values in the same index. This might further be advantageous for higher n which might lead to a higher number of empty partitions. With the salt information of the partition identifier, empty partitions of different partitions are different.

FIG. 8 shows a method for generating an n-Hamming search index value for a record (string), i e. an index value for an indexed database allowing an n-Hamming distant search in the index of the indexed database. This method realises for a record string stored in a record of the database the following steps.

In a first step S11, n+1 hash values are calculated for the record string as described in more detail in the method of FIG. 7. That means that for an n-Hamming distant search allowing n errors in the search, n+1 hash values are calculated or used, i.e. one hash value more than allowed errors in the search or in the retrieved record strings. The size of the hash values resulting from the used hash function can be selected based on the application. The longer the hash value, the more unlikely are occasional collisions between hash values of partitions with unequal symbol sequences. However, the longer the hash value, the more space is needed for the n-Hamming search index storing the n+1 hash values for each of the records of the database. For example, for an example database of 2 billion records with 3 hash values per record, results in a 3-Hamming search index of a size of 100 Gigabyte for a hash value of size 64 bit and in a 3-Hamming search index of a size of 50 Gigabyte for a hash value of size 32 bit. While the 64 bit hash values make a collision very unlikely, with a 32 bit hash value, there will be certain number of occasional hash collisions. Therefore, depending on the priority of speed or storage and depending on the number of records stored and based on the number n of errors of the n-Hamming distant search, the correct size of the hash value must be selected.

In a second step S12, the n+1 hash values are stored in association with the record (string) for which the n+1 hash values have been calculated. The n+1 hash values are stored in an indexed storage which facilitates searching for the hash value(s) and thus for the record associated with the hash value(s). The indexed storage comprises at least one index in which the n+1 hash values are stored in association with the record. Storing the index value might mean to add the index value in the indexed storage, if there is not yet any record with this index value (e.g. for a btree). However, for other indexed storages like hash tables, all possible index values have already been pre-generated and the storing of the index value in the index means just to associate the record with the respective index value in the index. Preferably, the (index of the) indexed storage comprises n+1 sub-indices, wherein each of the n+1 hash values are stored in a different one of the n+1 sub-indices. The i-th hash value Hi is stored in the i-th sub-index of the indexed storage with i=1, 2, . . . , n+1. This allows to quickly search for identical record strings by searching records having all n+1 hash values identical to the n+1 hash values of the query string. However, it could also be possible to store all n+1 hash values in the same index. To avoid collisions between different partitions (also of different record strings) having the same character sequence, the hash function in step S2 can be salted with an identifier of the partition of the record string (as salt information). That means that each partition has a different salt added to the partition. The identifier could be the partition number i or any other identifier distinguishing the n+1 different partitions of the record string. Consequently, each record of the database is associated to n+1 record hash values stored in an index of the indexed storage. If two partitions of the same record string or of different record strings have identical character/symbol sequences, the two hash values corresponding to the partitions with the same symbol sequence are equal, if there is no differentiating salt information. If the hash values of different partitions are stored in the same sub-index, the record(s) is/are associated thus twice to the same hash value. This would still work but increases the number of collisions significantly. Therefore, it is preferred to store the different hash values of different partitions in different sub-indices of the n-Hamming search index or to calculate the different hash values of different partitions with a different salt information which is different for each of the n+1 partitions, when the n+1 hash values of a record are stored in the same (sub-) index of the n-Hamming search index.

In an optional but preferred step S13, the length of the record string is stored in a further sub-index of the (n-Hamming search index of the) indexed storage. This allows to exclude already all record strings from the search which are of different length than the query string. In an alternative embodiment or also in addition to the sub-index for the length of the record string, the length of the record string could be used as salt information.

The method for generating an n-Hamming search index for a database generates for the record strings of each record of the database a n-Hamming search index value as described in FIG. 8. The n-Hamming search index values of all records are stored in the same index which is the n-Hamming search index. The n-Hamming-hash-index comprises preferably the n+1 sub-indices storing the n+1 hash values and/or the one sub-index for the length of the record string. However, as described above, the n-Hamming hash-index can comprise also less or more sub-indices or also comprise just one index. Each index value to which a record has been associated or which has been stored in the n-Hamming search index has an association to a record. The n-Hamming search index is quickly searchable due to its ordered storage so that the relevant records for the search can quickly be identified.

An existing database can be upgraded with an n-Hamming search index by generating for the record strings of all records an n-Hamming search index value stored for all records in the same n-Hamming search index. An index database with such an n-Hamming search index shall also be called an n-Hamming search database. For every new record stored in the n-Hamming search database, an n-Hamming search index value will be generated as describe in FIG. 8 and stored in the n-Hamming search index of the database.

If a database comprises more than one record string per record, there could be an n-Hamming search index as described above for each record string of the record. If the database comprises in each record a first record string and a second record string, the indexed database could comprise a first n-Hamming search index for the first record strings and a second n-Hamming search index for the second record strings. The different n-Hamming search indexes could have equal n or different n, depending on the application. It is also possible that the indexed database comprises for the same record string of the records a first n-Hamming search index and a second 1-Hamming search index with n and 1 being different. This would allow for example also search for the n-hamming distant search for the first record string with respect to a query string and for simple length search for the second and/or third record string of the same record.

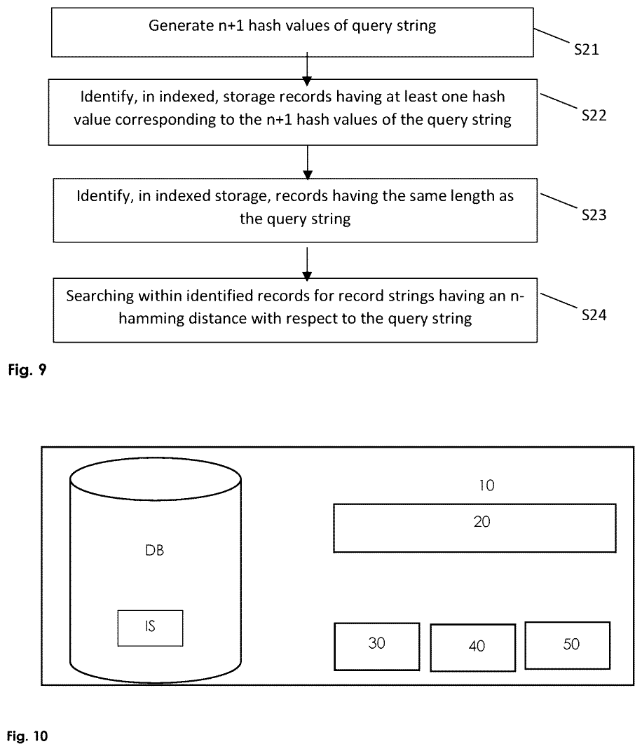

FIG. 9 shows the steps for performing an n-Hamming distant search in a database with an n-Hamming search index. The n-Hamming distant search allows to perform a search for a query string QS in the (first) record strings RS of the records of the database with a search condition, wherein the search condition is that the record strings RS of the resulting records RS have a Hamming distance smaller than or equal to n with respect to the query string QS or a more limited search condition. The broadest possible search condition allows thus to retrieve all records with a record string RS having a Hamming distance smaller than or equal to n with respect to the query string QS. A more limited search condition is any search condition which limits the search with a more limited search condition than record strings having a Hamming distance smaller than or equal to n with respect to the query string QS Such a more limited search condition could be that the resulting records correspond to (all) records having a Hamming distance with respect to the query string QS equal to n or equal to 1 or smaller than or equal to 1 with 1 being smaller than n. The more limited search condition could be also an additional condition for the record, e.g. for the record string or also for the other information stored in the record. An example more limited condition with an additional condition could be to search for all record strings RS having a Hamming distance smaller than or equal to n with respect to the query string QS and starting always with the three characters “QCM”. In the example below, this would further reduce the resulting records to only record R=6 because the record R=8 starts with “QCL”. The additional condition could however also be a condition for a further record string stored in the same record. The search condition can be configured by the user. This can be realized by a selection of the user among different search conditions. This can also be realized by allowing the user to program the search condition himself or herself. However, the user cannot extend the search condition beyond the condition that the record strings have a Hamming distance with respect to the query string QS smaller than or equal to n, because the n-Hamming search index does not allow to search (via the n-Hamming search index) for such search conditions, e.g. having a 1-Hamming index with 1 being larger then n or having a Levenshtein distance.

The steps of the search will be explained with the help of two examples.

A first illustrative example corresponds to the query string QS in FIG. 1 and a database with a 2-Hamming search index as shown in FIG. 2. The first eight records of the database are designed to be very similar to QS with the differences being marked in bold. For example, the records R=1, 2 correspond to the query string with an insertion at the end or the beginning. The record R=3 corresponds to the query string QS with three edits. The records R=4,5 correspond to the query string QS with a delete at the end and the beginning, respectively. The record string R=6 is identical to the query string QS. The record R=3 corresponds to the query string QS with three edits. The record R=8 corresponds to the query string QS with two edits. Thus, the records R=1, 2 are of length 15, the records R=4, 5 are of length 13 and the query string and the record strings of the records R=3, 6, 7 and 8 have the same length 14 of the query string.

A second example is a realisation with a database with 2 billion entries, wherein each entry comprises a CDR-H3 protein string in a FASTA format. The database comprises a 2-Hamming search index. The CDR-H3 protein strings have mostly a length between 10-30 characters. The number of symbols of the alphabet is 20. The partition function of FIG. 4 was used and a SHA-1 was used as hash function resulting each time in a 64-bit hash value. Preferably, the database comprises in the first record string a CDR-H3, in the second record string a CDR-H2 and in the third record string a CDR-H1. The database has also a second 2-Hamming search index for the second record string and a third 2-Hamming search index for the third record string. The 2-Hamming search index of the second and third record string corresponds to the first 2-hammind search index for the first record string, just that for the first 2-Hamming search index a 64-bit hash value has been used for the hash function, while for the second and third 2-Hamming search index a 32-bit hash value has been used. Since most searches include a search in the first record string, i.e. the CDR-H3, a faster search can be made due to the reduced number of collisions. Due to the reduced importance of the CDR-H1 and CDR-H2, a smaller space for storing the 2-Hamming indices for the second and the third record string seemed more advantageous than the speed of the search in the second and third record strings.

In a first step S21, n+1 hash values are calculated for the query string as

described in more detail in the method of FIG. 7 with k=n+1. That means that for an n-Hamming distant search allowing n errors in the search, n+1 hash values are calculated or used, i.e. one hash value more than allowed errors in the search. The same method for calculating the n+1 hash values is used as for calculating the n+1 hash values of each record of the database. This is important as the same record strings and/or partitions will lead to the same hash value(s). It is not important that the same method is used for different partitions, but it is important that for the same partition of different record strings and the query string, the same hash function is used. In the first and second example, with n=2, k=3 hash values are calculated. In the shown first example of FIG. 1, the query string QS has the three hash values H1=103, H2=144 and H3=988.

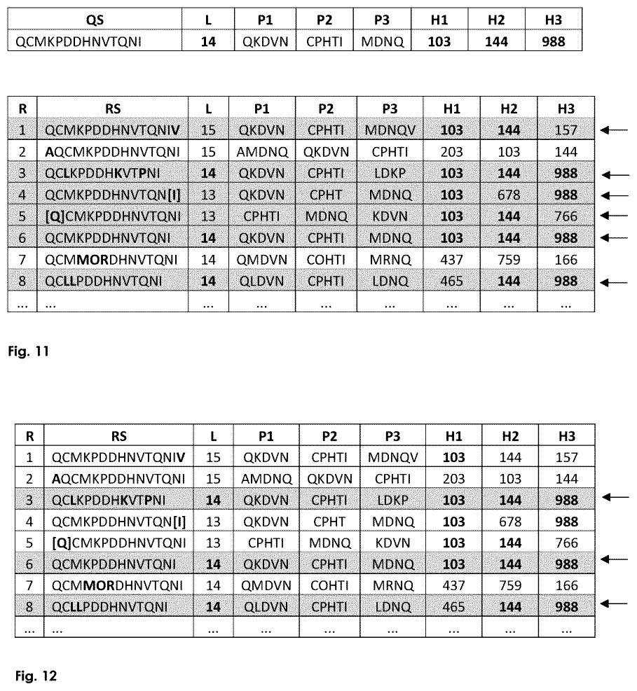

In a step S22, all the records of the database having at least one of the n+1 hash values identical to the corresponding hash value of the query string are identified. These identified records 1, 3, 4 5, 6 and 8 are shown in the first example in FIG. 11 which highlights the 6 identified records with a grey colour and/or with an arrow. In records R=1, 3, 4, 5, 6, the first hash value H1 corresponding to the first partition of the record string is equal to the first hash value H1 of the query string QS. In records R=1, 3, 5, 6, 8 the second hash value H2 corresponding to the second partition of the record string is equal to the second hash value H2 of the query string QS. In records R=3, 4, 6, 8 the third hash value H3 corresponding to the third partition of the record string is equal to the third hash value H3 of the query string QS. The comparison is preferably done only within the same hash value or the same partition such that a query string QS with a first partition P1 or first hash value H1 being equal to a second or third partition/hash value of the record string, would not be identified, if not another hash value in the same “category/partition” is identical. For example, in record R=2 of FIG. 11, the second hash value H2 corresponds to the first hash value H1 of the query string QS and the record R=2 is not identified in the sense of step S22. This can be achieved by creating different sub-indices per hash value, i.e. n+1 subindices (as shown in the example of FIGS. 2, 11 and 12 with 3 sub-indices H1, H2 and H3) and search the n+1 hash values only in their respective sub-indices. This means that the i-th hash value of the query string QS is only searched in the i-th subindex, so that (only) records are identified which have in the i-th sub-index an identical hash value as the i-th hash value of the query string QS. The i-th sub-index corresponds obviously to the i-th hash value of the record strings. In an alternative embodiment, all n+1 hash values of the record strings are stored in the same index, but the n+1 hash values have a salt information based on the identifier of the hash value or partition so that the hash values of an i-th partition and a j-th partition (with i unequal to j) comprising the same character/symbol sequence would lead to different hash values due to the salt information.

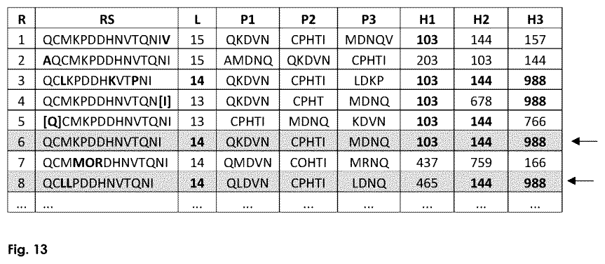

In a step S23, all records among the records identified in step S22 are identified having the same length as the query string. This is realized here by having a further n+2-th sub-index with the length of the record string which can be used to identify the records with the same length as the query string QS. FIG. 12 highlights the records R=3, 6, 8 among the records R=1, 3, 4, 5, 6, 8 having the length 14 of the query string QS. The step S23 is optional. The step S23 allows to further reduce the search space for step S24, because records having one or more identical partitions, but not having identical record string length are excluded as well from the identified records. This speeds up the n-Hamming distant search. However, also without step S23, the present n-Hamming distant search provides a significant acceleration compared to a search which compares the record strings of all records string by string.

The steps S22 and S23 can be performed in any order or also in their combination For example, the length of the record strings (and of the query string) could be used as salt information for all of the n+1 hash values so that partitions are only identical, if the partitions are equal and the strings have equal length (except for accidental collisions obviously). In such an embodiment, the step S23 is integrated in S22. In many indexed databases, a plurality of indexes can be searched cumulatively by cumulating them by logical operators. In the case n+1 sub-indices for the n+1 hash indices and one sub-index for the length, the n+1 hash value and the one length could be searched in the n+2 subindices by the following logical combination (H1 OR H2 OR . . . OR Hn+1) AND L, wherein Hi is the i-th hash value of the record strings and/or the query string with i=1, . . . , n+1 and L is the length of the record string and/or the query string. In the second example, a relational database was used which allows such a logical combination of sub-indices. It was discovered that in some cases, S23 or the search for the length was performed first before performing S22 or the search for the hash values. In other cases, vice versa. Thus, in a preferred embodiment, it is the database itself which decides in each case which sub-index is searched first to perform the index search most efficiently.

The identification step S22 (and also S23) is based on an indexed search. The identified records are identified by search through the n-Hamming search index. Due to the ordered arrangement of the n-Hamming search index, the identification step S22 (and also S23) can be realized very quickly and can reduce the further search space for the n-Hamming distant search. Due to the identification condition of S22, every record string having n+1 different hash values have n+1 different partitions than the query string and have thus more than n errors. The identification of records of steps S22 (and S23) or the indexed search exclude thus all records whose record string cannot have a Hamming distance equal to or smaller than n with respect to the query string QS. However, it cannot be guaranteed that all identified records indeed have a Hamming distance equal to or smaller than n with respect to the query string QS. Thus, the identification step S22 (and/or S23) (or the indexed search) allows just a search space reduction, but not an exact search result. For example, the identified record R=3 has actually a 3-Hamming distance with respect to the query string QS, because one partition contained two substitutions.

Therefore, it is necessary in a step S24 to search through the identified records for records fulfilling the search condition. In a preferred embodiment, the search through the identified records for records fulfilling the search condition is done by a search within the record strings of the identified records. For each identified record, it is checked, if the record fulfils the search condition. This includes, if the record string has a Hamming distance smaller than or equal to n with respect to the query string QS (broadest Hamming condition) or a more limited Hamming condition. A more limited Hamming condition is a condition which excludes at least one Hamming distance smaller than or equal to n. We will use here the term Hamming condition where we do not want to distinguish between the broadest Hamming condition and the more limited Hamming condition. Each search condition must comprise such a Hamming condition. The algorithm checking, if the record string of the respective identified record fulfils the Hamming condition of the search condition, receives as input the query string and the record string of the respective identified record and gives out an output indicating, if the Hamming condition is fulfilled or not. Thus, only with step S24, it can be assured that the resulting records all fulfil the search condition. Thus, the step S24 excludes in the first example the identified record with the record string RS with a Hamming distance of 3, thus larger than 2 and results in the final records R=6 and 8 which fulfil the search condition which in this case is the broadest Hamming condition with n=2. FIG. 13 highlights the resulting records R=6, 8 among the records R=3, 6, 8 fulfilling the search condition. In the example, the 2-Hamming distant search in the database with 2 billion records was performed around a second, while searches comparing each record string by string take several hours to perform the same search. In another embodiment, it is also possible to apply other search algorithms for searching through the identified records. This can include also the application of further partitioned hash searches with different n.

This is a huge advantage for the research of antibodies where one needs to identify similar proteins in large databases. Since many of these searches are required, a search time of several hours slows the research significantly down. The application for hash values on partitions for short biological amino acid sequences like for proteins, in particular for CDR-H3 are very new for the purpose of performing an n-Hamming distant search. This search tool can significantly accelerate antibody research based on CDR-H3 of proteins.

The n-Hamming search index can further be used to search for identical strings, i.e. Hamming distance of zero. This can be realized by identifying all records whose n+1 record hash values correspond to the n+1 query hash values. In the case of n+1 sub-indices among which the n+1 query hash values are searched, the n+1 sub-index-searches can be combined by a logical AND so that the result will yield only the records with a record string identical to the query string. In this special case, the step S24 is not necessary anymore.

FIG. 10 shows an embodiment of a system 10 for performing a n-Hamming distant search. The system comprises a database DB and a processing means 20.

The database DB is preferably an indexed database with an index IS. The index is preferably the n-Hamming search index as described above. The database DB is preferably stored in a non-volatile storage means.

The processing means 20 is configured to perform the n-Hamming distant search as described above on the records of the database DB and/or on its index IS. Preferably, the processing means 20 comprises one or more processors, e.g. CPUs. A plurality of processors can be combined in the same chips, like in multi-core CPUs or via a network of parallel CPUs or via a cloud computing network. However, it is also possible to realize the search on one single processor. Preferably, the processor is a general processing unit loading a software program from a program storage 30 into an internal, preferably quick and/or volatile storage like a RAM, where it can be executed by the processing means 20. However, the processing means 20 can also be realized as a special purpose chip or processing means designed for this special task of performing the n-Hamming distant search.

The system comprises preferably further the internal storage 40. The internal storage 40 is preferably configured to execute the computer program for the n-Hamming distant search and/or for loading the n-Hamming search index IS in the internal storage. Therefore, the internal storage 40 must be large enough to store/load the complete n-Hamming search index IS. This will accelerate the n-Hamming distant search significantly as the memory for the index search operations on all records are all performed in the internal storage 40. Preferably, the n-Hamming search index IS is kept in the internal storage 40 as long as the program for the n-Hamming distant search is running so that each n-Hamming distant search request can be performed quickly. However, due to the significant reduction of the run time of the n-Hamming distant search, the n-Hamming distant search could also be performed a bit more slowly with the n-Hamming search index stored only in the database.

The system 10 comprises preferably further a user interface 50. The user interface is preferably configured to output the resulting records to a user, e.g. on a display, in a file, in a message over a network interface. Therefore, the user interface can comprise a monitor, a data or network interface. The user interface is preferably configured to receive user input. The user input is preferably configured to make a search request for an n-Hamming search a query string QS or to define the search condition of the search request. The user interface can comprise a keyboard, a mouse or any other user input means. The user input means also be a front end, e.g. when the processing means is a server which receives the user input over a front end. The user interface 50 can also be an application interface (API) to allow to input and/or output information via the API.

When the strings stored in the database comprise also indirect letters, there are different solutions to this problem. Either, the same string is translated in a plurality of strings comprising only direct symbols, wherein the plurality of strings covering all possible realisations of the string with at least one indirect letter. Each of the plurality of strings is then stored as an independent entry or at least the n has values of each of the plurality of strings are stored in the indexed storage referring to the same entry. It is however also possible to treat the case where the same character position of a record string and a query string comprise at least once an indirect letter which depending on the realisation of the at least one indirect letter could be identical is treated simply as an error.

While some strings can be read only in one direction, other strings maybe read in different directions. For example, amino acids are read normally only from left to right. On the other side, nucleotide sequences can be read either from left to right (forward direction) or from right to left (reverse direction) or in complement (translating A, T, G, C into their respective complement bases T, A, C, G) or in reverse complement (reverse direction and complement). So, in one embodiment, the string has (only) one consecutive order and the query string and the record string are always compared in the same consecutive order. In another embodiment, the string can have at least two consecutive orders. In this case, the user could select in the query the consecutive order in which he wants to search for the query string, from left to right (standard) or from right to left (reverse), or complement or reverse complement. In the letter case, the query string would then be inverted, complemented or reverse complemented before creating the partitions and hashes. It would also be possible to search for two or more consecutive orders which could be realised by two or more searches, a first search for the query string in a first consecutive order (a first one of forward, reverse, complement and reverse complement), a second search for the query string in a second consecutive order (a second one of forward, reverse, complement and reverse complement), maybe a third search for the query string in a third consecutive order (a third one of forward, reverse, complement and reverse complement) and maybe a fourth search for the query string in a fourth consecutive order (a third one of forward, reverse, complement and reverse complement).

It should be understood that the present invention is not limited to the described embodiments and that variations can be applied without going outside of the scope of the claims.

1. Method for performing an n-Hamming distance search in a database, wherein the database comprises a plurality of records and an indexed storage, wherein each record comprises a record string, wherein each record of the database is associated to n+1 record hash values stored in an index of the indexed storage, wherein the method comprises the following steps:
partitioning a query string into n+1 query partitions, wherein the n+1 query partitions are pairwise disjoint;
creating a hash value for each query partition resulting in n+1 query hash values;
identifying records having at least one record hash value equal to one of the n+1 query hash values resulting in identified records; and
searching within the identified records for resulting records fulfilling a search condition, wherein the search condition is that the record strings of the resulting records have a Hamming distance smaller than or equal to n with respect to the query string or a more limited search condition.

2. The method according claim 1, wherein a user can select or program different search conditions as the more limited search condition.

3. The method according claim 1, wherein the searching within the identified records for resulting records is performed with an algorithm which works on the record strings of the identified records to verify the search condition.

4. The method according claim 1, wherein the alphabet of the query string and the record strings comprises twenty or more different symbols.

5. The method according claim 1, wherein the length of the record strings is smaller than hundred characters.

6. The method according claim 1, wherein the query string is partitioned such that at least one of the n+1 partitions comprises a non-consecutive character sequence of the query string.

7. The method according claim 1, wherein the hash value for each partition is created by applying a hash function on the partition.

8. The method according claim 1, wherein the hash value for each partition is created by applying a hash function on the combination of the partition and a salt information.

9. The method according claim 1, wherein the step of identifying records having at least one record hash value equal to one of the n+1 query hash values resulting in identified records corresponds to identifying records having for at least one i being a natural number between 1 and n+1 the i-th hash value of the record string equal to the i-th hash value of the query string as the identified records.

10. The method according claim 1, wherein the identified records are identified as the records cumulatively having at least one record hash value equal to one of the n+1 query hash values resulting in identified records and having the same length as the query string.

11. The method according claim 1, wherein the records of the database are stored in an indexed storage with the n+1 hash values of the records working as indices for the indexed storage, wherein the identified records are identified by searching the n+1 query hash values in the respective n+1 hash value indices.

12. The method according to claim 11, wherein the length of the record string is a further index of the indexed storage.

13. The method according claim 1, wherein each record comprises a Protein.

14. The method according to claim 13, wherein the protein is an antibody, a T-cell receptor or a B-cell receptor.

15. The method according claim 1, wherein the record string is an amino acid sequence.

16. The method according claim 1, wherein the record string is a complementarity-determining region of a protein.

17. A method for generating an n-Hamming search index for a database allowing an n-Hamming distance search in the database, the method comprises for each record of the database the following steps:
partitioning the record string into n+1 record partitions, wherein the n+1 record partitions are pairwise disjoint;
creating a hash value for each record partition resulting in n+1 record hash values; and
storing the n+1 record hash values as n-Hamming search index value for the record in the n-Hamming search index.

18. A non-transitory computer program for performing an n-Hamming distance search in a database, the non-transitory computer program comprising instructions, when executed on a processor, configured to perform in the processor the steps:
wherein the method comprises the following steps;
partitioning a query string (QS) into n+1 query partitions, wherein the n+1 query partitions are pairwise disjoint;
creating a hash value for each query partition resulting in n+1 query hash values;
identifying among a plurality of records of a database records having at least one record hash value equal to one of the n+1 query hash values resulting in identified records, wherein each record of the database comprises a record string, wherein each record of the database is associated to n+1 record hash values stored in an index of the database; and
searching within the identified records for resulting records fulfilling a search condition. wherein the search condition is that the record strings of the resulting records have a Hamming distance smaller than or equal to n with respect to the query string or a more limited search condition.

19. A non-transitory database with an n-Hamming search index allowing an n-Hamming distance search, the database comprising a plurality of records, wherein each record comprises a record string and an n-Hamming search index value, wherein the n-Hamming search index value of the respective record comprises n+1 record hash values, wherein the n+1 record hash values correspond to the hash values of n+1 record partitions of the respective record, wherein the n+1 record partitions of the respective record are pairwise disjoint, wherein n-Hamming search index is constituted by the n-Hamming search index values of the records.

20. (canceled)

21. (canceled)
